(19) United States
(12) Patent Application Publication
(10) Pub. No.: US 2026/0013758 A1
(43) Pub. Date: Jan. 15, 2026

(54) CALIBRATION FOR A BIOLOGICAL SENSOR

(71) Applicant: Allez Health Inc., Carlsbad, CA (US)

(72) Inventors: Robert James Boock, Carlsbad, CA (US); Sai Kit Wu, Irvine, CA (US)

(73) Assignee: Allez Health Inc., Carlsbad, CA (US)

(21) Appl. No.: 19/264,592

(22) Filed: Jul. 9, 2025

Related U.S. Application Data
(60) Provisional application No. 63/669,856, filed on Jul. 11, 2024.

(51) Int. Cl. A61B 5/1495

(52) U.S. Cl. CPC A61B 5/1495; A61B 2560/0204; A61B 2560/0223

(57) ABSTRACT

Calibration for a biological sensor involves a holder coupled to a housing and configured to retain a wire-holding fixture that holds a working wire for the biological sensor. An electrical test board is coupled to the housing, wherein the electrical test board is configured with electronics of a continuous biological monitor and has an electrical contact configured to electrically connect to the working wire through the wire-holding fixture.

### RELATED APPLICATIONS

This application claims priority to U.S. Provisional Patent Application No. 63/669,856, filed on Jul. 11, 2024, and entitled “AUTOMATED CALIBRATION FOR A BIOLOGICAL SENSOR”; the contents of which are incorporated herein by reference in full.

### BACKGROUND

Monitoring of glucose levels is critical for diabetes patients. Continuous glucose monitoring (CGM) sensors are a type of device in which glucose is measured from fluid sampled in an area just under the skin multiple times a day. CGM devices typically involve a small housing in which the electronics are located and which is adhered to the patient's skin to be worn for a period of time. A small needle within the device delivers the subcutaneous sensor, which is often electrochemical.

The CGM sensor is typically temporarily adhered to the patient's skin with an adhesive pad, and the CGM sensor couples to a small housing in which electronics are located. The CGM sensor typically has a disposable applicator device that uses a small introducer needle to deliver the CGM sensor subcutaneously for the patient. Once the CGM sensor is in place, the applicator is discarded, and the electronics housing is attached to the sensor. Although the electronics housing is reusable and may be used for extended periods, the CGM sensor and applicator need to be replaced often, usually every few days.

Electrochemical glucose sensors operate by using electrodes which typically detect an amperometric signal caused by oxidation of enzymes during conversion of glucose to gluconolactone. The amperometric signal can then be correlated to a glucose concentration. The electrode typically includes an electrically conductive substrate and one or more membrane layers that produce electrical current in response to the amount of glucose present in the patient's body. The construction of these layers affects the performance of the device, such as the output readings and sensitivity. Manufacturing processes for CGM sensors can affect the accuracy and repeatability of fabricating these layers. Proper assembly, testing, and calibration of the CGM device are also important for accurate operation of the device in the field.

### SUMMARY

In some aspects, the techniques described herein relate to an apparatus for calibrating a biological sensor, including: a housing; a holder coupled to the housing, the holder configured to retain a wire-holding fixture for holding a working wire for the biological sensor; and an electrical test board coupled to the housing, wherein the electrical test board is configured with electronics of a continuous biological monitor and has an electrical contact configured to electrically connect to the working wire through the wire-holding fixture.

In some aspects, the techniques described herein relate to a system for calibrating a biological sensor, including: a plurality of containers configured to hold calibration solutions; a mobile calibration apparatus configured to be placed on the containers; a wire-holding fixture for holding a working wire for the biological sensor; and a robot assembly configured to transport a combination of the mobile calibration apparatus and the wire-holding fixture to and from the plurality of containers for a calibration process; wherein the mobile calibration apparatus includes: a housing; a holder coupled to the housing, the holder configured to hold the wire-holding fixture; and an electrical test board coupled to the housing, wherein the electrical test board is configured with electronics of a continuous biological monitor and has an electrical contact configured to electrically connect to the working wire through the wire-holding fixture.

In some aspects, the techniques described herein relate to a method including: providing a combination of a mobile calibration apparatus and a wire-holding fixture, wherein the wire-holding fixture holds a working wire for a biological sensor; transporting, by a robot assembly, the combination of the mobile calibration apparatus and the wire-holding fixture through a sequence of containers containing calibration solutions, wherein the combination of the mobile calibration apparatus and the wire-holding fixture is sequentially placed above each container in the sequence with the working wire extending into the calibration solution; testing the working wire with the calibration solutions; recording calibration responses of the working wire with respect to the calibration solutions; and calibrating the biological sensor based on the recorded calibrated responses; wherein the mobile calibration apparatus includes: a housing; a holder coupled to the housing, the holder configured to hold the wire-holding fixture; and an electrical test board coupled to the housing, wherein the electrical test board is configured with electronics of a continuous biological monitor and has an electrical contact configured to electrically connect to the working wire through the wire-holding fixture.

### BRIEF DESCRIPTION OF THE DRAWINGS

FIG. 1 is a not-to-scale cross-sectional view of a working wire, in accordance with some examples.

FIGS. 2A and 2B show a first side and second side of a wire-holding fixture, in accordance with some examples.

FIGS. 3A-3C are views of a mobile calibration apparatus for calibrating working wires of a biological sensor (and thus for calibrating the biological sensor), in accordance with some examples.

FIGS. 4A and 4B are views of a calibration system for calibrating a working wire of a biological sensor (and thus for calibrating the biological sensor), in accordance with some examples.

FIG. 4C is a perspective of a robot assembly in the calibration system of FIGS. 4A and 4B, in accordance with some examples.

FIGS. 5A and 5B are views of a fixture tray in a holding area of the calibration system of FIGS. 4A and 4B, in accordance with some examples.

FIG. 6 is a perspective view of a robotic manipulation arm of the robot assembly of FIG. 4C, in accordance with some examples.

FIGS. 7A and 7B are perspective views of a calibration array of the calibration system of FIGS. 4A and 4B, in accordance with some examples.

FIG. 8 is a flowchart of an example calibration process for calibrating a working wire for a biological sensor (and thus for calibrating the biological sensor) in an automated manner, in accordance with some examples.

FIG. 9 is a simplified schematic diagram showing an example computer for use in the calibration system, in accordance with some examples.

### DETAILED DESCRIPTION

Systems and processes for manufacturing, testing and calibrating working wires for a continuous biological sensor or continuous biological monitoring device are described herein. The systems and processes enable manufacturing scalability and improve accuracy and efficiency compared to known art. The continuous biological sensor may be, for example, a continuous glucose monitor, in which the working wire includes an enzyme layer to detect the level of glucose in a patient's blood. In other examples, the biological sensor can be a metabolic sensor for measuring other metabolic characteristics such as lactate, ketone or fatty acids. The sensor uses a working wire (i.e., an electrode for the sensor) that has a core and several concentrically formed membrane layers on the core.

The systems and processes described herein enable automated calibration of a working wire for a biological sensor and thus the calibration of the biological sensor, such as a glucose sensor for a continuous glucose monitoring device or a lactate sensor for a continuous lactate monitoring device. The systems and processes uniquely provide automated testing of biological sensors in an efficient and repeatable manner, and furthermore provide tracking of each individual wire that is processed. This traceability is advantageous not only for keeping manufacturing records, but also enables precise calibration of each individual sensor wire and consequently the sensitivity of the final biological sensor. This ability to correlate an individual biological sensor's behavior over time can eliminate or reduce the need for local calibration by patients through finger stick monitoring, as is done with conventional CGM sensors.

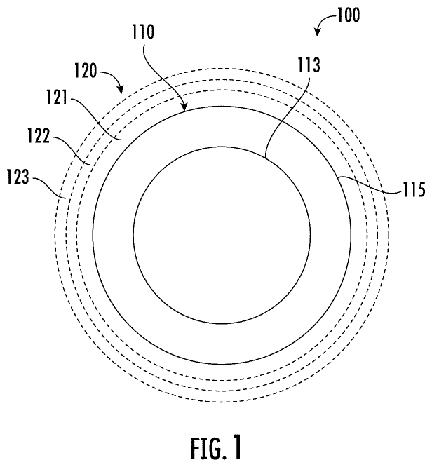

Referring to FIG. 1, a cross-sectional view of a working wire 100 is illustrated in accordance with some examples. In this example, the working wire 100 is an elongated wire having a circular cross-section. It will be understood that other cross-sections may be used, such as square, rectangular, triangular, or other geometric shapes. Furthermore, the working wire 100 may take other forms, such as a plate or ribbon. The working wire is used as a working electrode of a continuous biological sensor, such as a working electrode of a continuous glucose monitor.

In the illustrated example, the working wire 100 has a substrate 110 onto which biological membranes 120 may be disposed. In one example as illustrated, the biological membranes 120 include an interference membrane 121 (which may also be referred to as an interference layer) on the substrate 110, an enzyme membrane 122 (i.e., an enzyme layer) on the interference membrane 121, and a glucose limiting membrane 123 (i.e., a glucose limiting layer) on the enzyme membrane 122. In some examples, a protective or outer coating may be optionally applied over the glucose limiting membrane 123. In cases where the working wire 100 is for a lactate sensor, glucose limiting membrane 123 may be instead configured as a lactate limiting layer. Although the working wire 100 is illustrated as having three biological membranes 120, it will be understood that the biological membranes 120 may be more or fewer in number.

The substrate 110 may be comprised of a core 113 with an outer layer 115. In the example of FIG. 1, the core 113 is an elongated wire that is dense, ductile, very hard, easily fabricated, highly conductive of heat and electricity, and may also be resistant to corrosion. Example materials for core 113 include tantalum (Ta), carbon (C), or cobalt-chromium (Co—Cr) alloys. The core 113 may have the outer layer 115, such as of platinum (Pt), deposited or applied using an electroplating process. It will be understood that other processes may be used for applying the outer layer 115 to the core 113. For a glucose monitor, the platinum outer layer facilitates a reaction where hydrogen peroxide reacts to produce water and hydrogen ions, and two electrons are generated. The electrons are drawn into the platinum by a bias voltage placed across the platinum wire and a reference electrode. In this way, the magnitude of the electrical current flowing in the platinum is intended to be related to the number of hydrogen peroxide reactions, which in turn is proportional to the number of oxidized glucose molecules. A measurement of the electrical current on the platinum wire can thereby be associated with a particular level of glucose in the patient's blood or interstitial fluid (ISF) (a biological fluid in the patient's body that contains diverse biomarkers and analytes and is similar to blood composition).

The core 113, outer layer 115, interference membrane 121, and enzyme membrane 122 form key aspects of the working wire 100. Other layers and/or membranes may be added depending upon the biological substance being tested for, and application-specific requirements. In some cases, the core 113 may have an inner core portion (not shown). For example, if the substrate (core 113) is made from tantalum, an inner core of titanium (Ti) or titanium alloy may be included to provide additional strength and straightness for the working wire 100.

One or more membranes (i.e., layers) may be provided over the enzyme membrane 122. For example, the glucose limiting membrane 123 may be layered on top of the enzyme membrane 122. This glucose limiting membrane 123 may limit the number of glucose molecules that can pass through the glucose limiting membrane 123 and into the enzyme membrane 122. The glucose limiting membrane 123 can be configured as described in U.S. Pat. No. 11,576,595, entitled “Enhanced Sensor for a Continuous Biological Monitor,” which is owned by the assignee of the present disclosure and is incorporated herein by reference as if set forth in its entirety. In some cases, the addition of the glucose limiting membrane 123 has been shown to enable better performance of the overall working wire 100.

The interference membrane 121 is applied over the outer layer 115 of the substrate 110. The interference membrane 121 may be disposed between the enzyme membrane 122 and the outer layer 115. This interference membrane 121 is constructed to fully wrap the outer layer 115, thereby protecting the outer layer 115 from, or mitigating, oxidation effects. The interference membrane 121 is also constructed to substantially restrict the passage of larger molecules, such as acetaminophen, to reduce contaminants that can reach the platinum of the outer layer 115 and skew results. Further, the interference membrane 121 may pass a controlled level of hydrogen peroxide (H₂O₂) from the enzyme membrane 122 to the platinum outer layer 115. Compositions for the interference membrane 121 and the enzyme membrane 122 may be as described in U.S. patent application Ser. No. 19/047,285, entitled “Continuous Biological Sensor with Enzyme Immobilization,” and U.S. patent application Ser. No. 17/449,380, entitled “In-Vivo Glucose Specific Sensor,” which are owned by the assignee of the present disclosure and incorporated herein by reference as if set forth in their entirety.

The interference membrane 121 may be electrodeposited onto the electrical conducting wire (i.e., on the substrate 110) in a very consistent and conformal way, thus reducing manufacturing costs (e.g., compared to conventional sensor calibration processes or relative to non-electrochemical deposition methods, which may require more material, time, or complex equipment to achieve uniform coverage) as well as providing a more controllable and repeatable layer formation. The interference membrane 121 is formulated to be nonconducting of electrons but will pass negative ions at a preselected rate. Further, the interference membrane 121 may be formulated to be permselective (i.e., semipermeable) for particular molecules. In one example, the interference membrane 121 is formulated and deposited in a way to restrict the passage of larger molecules, which may act as contaminants to degrade the conducting layer (i.e., the substrate 110), or that may interfere with the electrical detection and transmission processes.

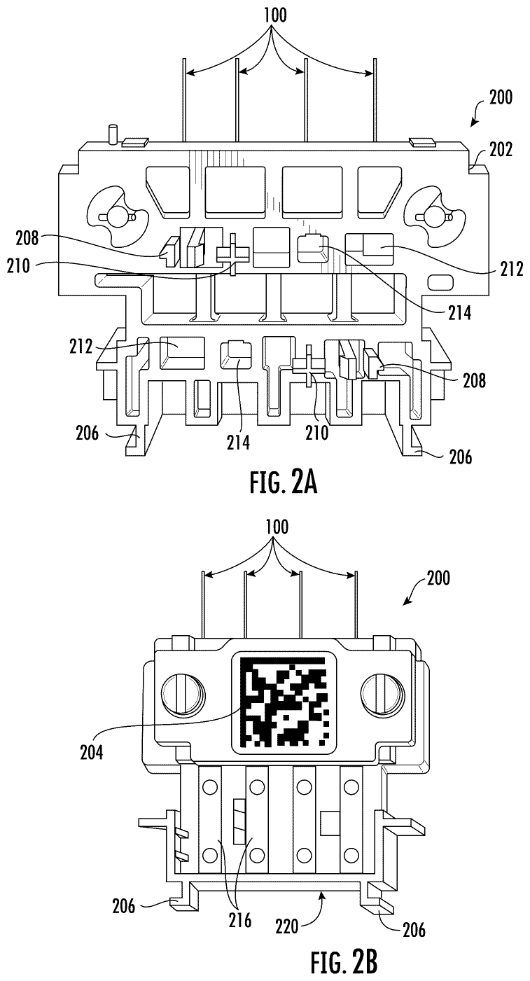

FIGS. 2A-2B show a first side and second side of a wire-holding fixture 200, in accordance with some examples. The wire-holding fixture is configured to hold a plurality of working wires for a corresponding plurality of biological sensors. In FIG. 2A, four working wires 100 are shown mounted into a body 202 of the wire-holding fixture 200. In other examples, the wire-holding fixture 200 may be configured to hold more or fewer working wires during manufacturing, such as 1 to 10, or 2 to 8, or 2 to 6, or other appropriate number or range. The wire-holding fixture 200 is a holder for transporting the working wires through various steps during manufacturing (e.g., electropolymerization coating, dipping, calibration, etc.). The wire-holding fixture 200 may also be referred to as a carrier or tray. The working wires may be secured into the wire-holding fixture 200 by, for example, clamps, spring-loaded clips, set screws, adhesive fasteners, or other mechanisms. The wire-holding fixture 200 may include an identifier 204 such as a scannable code (e.g., bar code or quick response “QR” code) for tracking the progress of the particular wire-holding fixture 200 during manufacturing. The identifier 204 may be other types of identifying labels, such as a radiofrequency identification (RFID) tag. The working wires 100 are mounted in a single row in this example, spaced apart and extending from an edge of the wire-holding fixture 200 so that each one can be measured individually from various angles. In other examples, the working wires may be arranged in other fashions, such as in more than one row, aligned or staggered from each other, as long as sufficient space is provided between the working wires to enable each wire to be measured or tested/calibrated separately. The working wires 100 extend away from the wire-holding fixture 200 to be able to extend downward into a container of a calibration solution when the wire-holding fixture 200 is placed in a calibration station (with the working wires 100 extending downwards) during a calibration process.

The wire-holding fixture 200 has L-shaped feet 206 to enable it to stand, be fixed in place, or be hung upside down as needed. Snapping tabs 208 are also included, extending outward from the face of the wire-holding fixture 200, to enable two wire-holding fixtures 200 to be snapped together (e.g., in pairs, back-to-back) and transported together during manufacturing and/or testing/calibration. In this illustration, two sets of snapping tabs 208 and alignment posts 210 are positioned diagonally from each other on the wire-holding fixture 200. When two wire-holding fixtures 200 are placed with the same sides facing each other, a snapping tab 208 on one wire-holding fixture 200 fits into a tab hole 212 on the other wire-holding fixture 200 that it is being attached to (i.e., a mating fixture), and the alignment post 210 fits into an alignment hole 214 of the mating fixture. The diagonal positioning of the alignment posts 210 on the wire-holding fixtures 200 helps ensure that the wire-holding fixtures 200 are properly aligned in all axes, and the snapping tabs 208 lock the wire-holding fixtures 200 together. Being able to thus “gang” or attach wire-holding fixtures 200 together allows more than one wire-holding fixture 200 to be moved together at a time (e.g., by a robot), which increases manufacturing speed. Various features of the wire-holding fixture 200 such as wings, indentations, grooves, and the like, may be used as gripping features for a robot to grab. The features may be configured to provide proper alignment in a robot (e.g., in x-y-z linear and rotational directions) and when inserted into a manufacturing process (e.g., dipping, coating, or electrical testing/calibration), since misalignment can affect the uniformity and/or concentricity of the coatings on the working wires 100 or electrical contact of the wire-holding fixture 200 with a test/calibration apparatus.

In FIG. 2B, the opposite face of the wire-holding fixture 200 is shown (with some optional variations). The identifier 204 (or other scannable code) is included for tracking the wire-holding fixture 200 during manufacturing and testing/calibration as described above. In this example, the QR code is a sticker that is attached to a plate 218 (e.g., metal) that is attached to the body 202 (e.g., plastic). Additionally, metal strips 216 are also shown attached to the body 202. One metal strip 216 is present for and electrically connected to a corresponding working wire 100 in the wire-holding fixture 200. The metal strips 216 provide an electrical connection between the working wire 100 and a terminal point on a bottom side 220 of the wire-holding fixture 200 for various manufacturing process steps, such as electropolymerization and electrical testing (e.g., calibration).

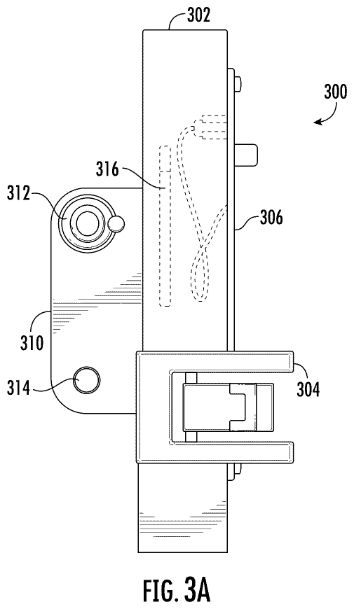

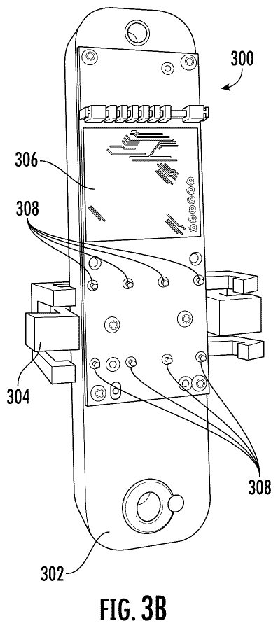

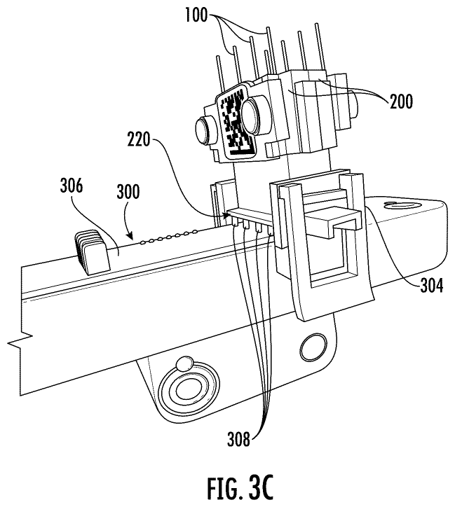

FIG. 3A is a side view and FIG. 3B is a front view of a mobile calibration apparatus 300 (which may also be referred to as a mobile apparatus or mobile applicator in this disclosure) for calibrating working wires 100 of a biological sensor (and thus for calibrating the biological sensor), in accordance with some examples. The mobile calibration apparatus 300 includes a housing 302, a holder 304 coupled to the housing 302, and an electrical test board 306 coupled to a surface of the housing 302. The holder 304 is configured to retain (i.e., hold, grasp) a wire-holding fixture 200 (FIGS. 2A-2B). For example, the holder 304 may be configured with tabs that snap into mating features (e.g., the feet 206) on the wire-holding fixture 200. Other types of coupling features may be utilized instead of or in addition to the tabs, such as friction fit mating features, latches, or magnets. FIG. 3C shows an example of the mobile calibration apparatus 300 having multiple wire-holding fixtures 200 inserted into the holder 304 of the mobile calibration apparatus 300. (In this example, two of the wire-holding fixtures 200 are snapped together and inserted into the holder 304, but any appropriate number may be used in other examples.) The metal strips 216 (see FIG. 2B) of the wire-holding fixtures 200 wrap around onto the bottom side 220 thereof. When multiple wire-holding fixtures 200 are coupled to the mobile calibration apparatus 300, electrical contacts 308 (e.g., pogo pins) of the electrical test board 306 contact the metal strips 216 at the bottom side 220 of the wire-holding fixtures 200.

The mobile calibration apparatus 300 of the present disclosure can be hand-held (i.e., manipulated by a system user) or moved by a robotic mechanism within the calibration system. In this example, a handle 310 (FIG. 3A) is coupled to or integrated on the housing 302. The handle 310 has two alignment features 312 and 314 configured to be gripped by a robotic manipulation arm. The first alignment feature 312 is a hole of a larger diameter than that of the second alignment feature 314 that is at an opposite end of the handle 310. By using the alignment features 312 and 314 with different dimensions, corresponding alignment features (e.g., posts or pins) on a robotic manipulation arm can ensure that the mobile calibration apparatus 300 is oriented properly (e.g., a front end of the housing 302 facing a desired direction) for picking up the mobile calibration apparatus 300 with the wire-holding fixture 200 and moving the wire-holding fixture 200 (or a fixture/apparatus combination, i.e., a combination comprising the mobile calibration apparatus 300 of FIGS. 3A-3C with the wire-holding fixtures 200 of FIGS. 2A and 2B attached thereto) through the calibration system and process. In other examples, the handle 310 may be omitted, and other alignment features may be incorporated on the body of the housing 302 itself (e.g., on the side surfaces of the housing 302). Although the alignment features 312 and 314 are shown as holes in this example, other configurations are possible such as male features instead of female features, different shapes (e.g., triangular, rectangular, linear instead of circular), or different numbers of features instead of two (e.g., one, three, or more).

The electrical test board 306 is configured with electronics of a continuous biological monitoring device or biological sensor and has at least one electrical contact 308 configured to connect to the wire-holding fixture 200. The electrical test board 306 simulates the electronic circuitry that will be used in a finished biological sensor device, thus ensuring accurate calibration of the working wires 100. The electrical contact 308 may be, for example, a spring-loaded pogo pin that enables quick connection and disconnection of the wire-holding fixture 200 to the mobile calibration apparatus 300. In this example, two rows of four electrical contacts 308 are included to accommodate two wire-holding fixtures 200 (e.g., snapped together as described in relation to FIG. 2A), each wire-holding fixture 200 holding four working wires 100 arranged in a row.

The housing 302 is hollow in this example, containing wiring for the electrical test board 306. Additionally, a battery 316 is included inside the housing 302 in this example. The battery 316 is electrically connected to the electrical test board 306 to provide power throughout the duration of the calibration process to the electrical test board 306 and to the working wires 100 that are coupled through the wire-holding fixture 200 to the mobile calibration apparatus 300. In some examples, the mobile calibration apparatus 300 is configured to recharge the battery 316 whenever the mobile calibration apparatus 300 is plugged into the calibration system, to ensure that the battery 316 will last during the entire calibration process. The battery 316 or other power source provides continuous electrical power to the working wires 100 during the entire period while the working wires 100 are calibrated, i.e., during the entire time in which the fixture/apparatus combination is transported through the sequence of containers for the calibration process.

The mobile calibration apparatus 300 is designed to keep the working wires 100 connected to a power source (e.g., the battery 316) during calibration, and to move the electronics with the working wires 100 during the process. In contrast, in conventional calibration techniques, an electrochemical break-in period must be restarted every time power is disconnected from a working wire. This break-in period can be lengthy, such as on the order of 30 minutes. Also in conventional techniques, each working wire has a physical wire connecting it to a main electrical board, which is cumbersome, difficult to automate, and creates crosstalk between wires. Thus, the mobile calibration apparatus 300 of the present disclosure can greatly reduce calibration time and the labor required, while also improving accuracy and efficiency.

In some examples, the electrical test board 306 applies electrical power to the working wires 100 and reads the responses thereto through the electrical contacts 308 to and from the working wires 100 in the wire-holding fixtures 200. The electrical test board 306 contains circuitry that has the same analog front end as the commercial wearable device (e.g., the finished biological sensor device) that will be used with the working wires 100, so that the results generated from the calibration system are as close as possible to the in vivo use. In general, the electrical test board 306 may be configured to power the working wires 100 and apply a bias voltage to amplify the sensor output to be readable. The electrical test board 306 may include, for example, an analog circuit having a transimpedance amplifier to amplify the current response generated from the working wire 100. The current response is typically on the order of picoAmps or nanoAmps, so the amplifier handles and converts the response into a signal that can be easily correlated.

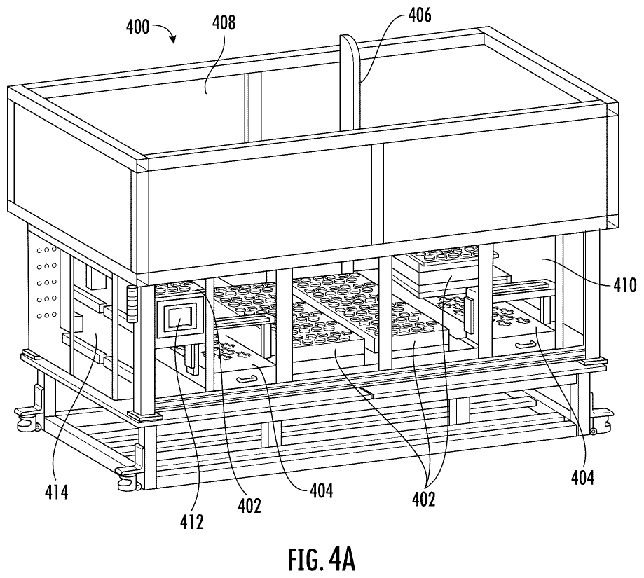

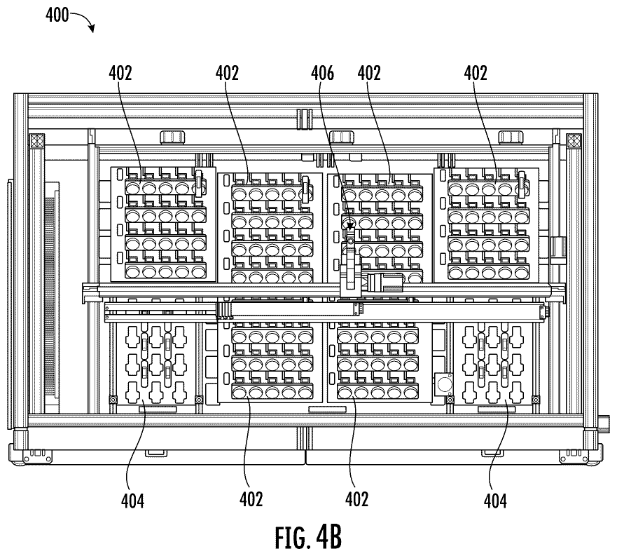

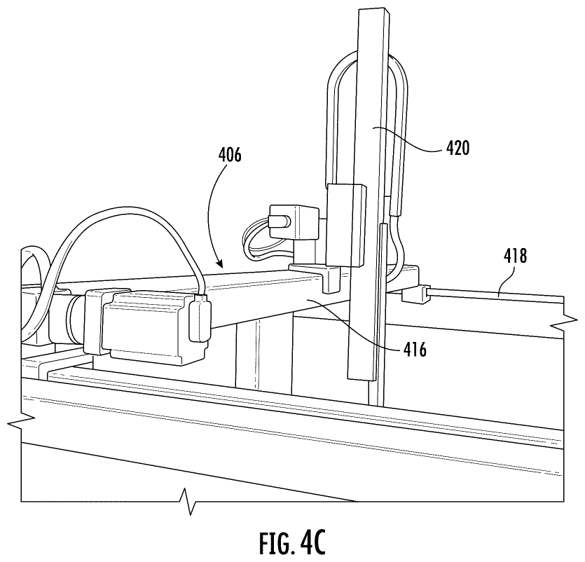

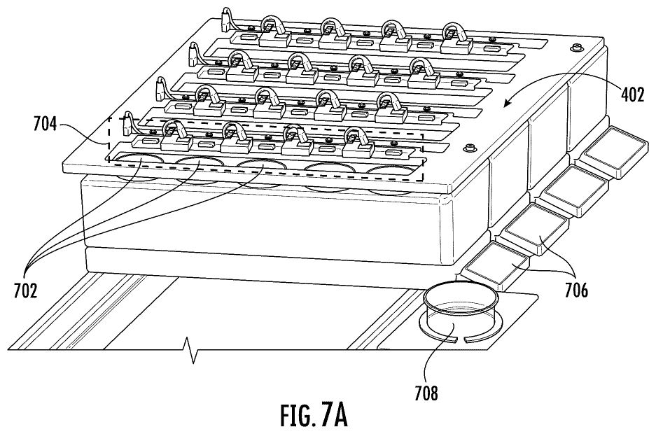

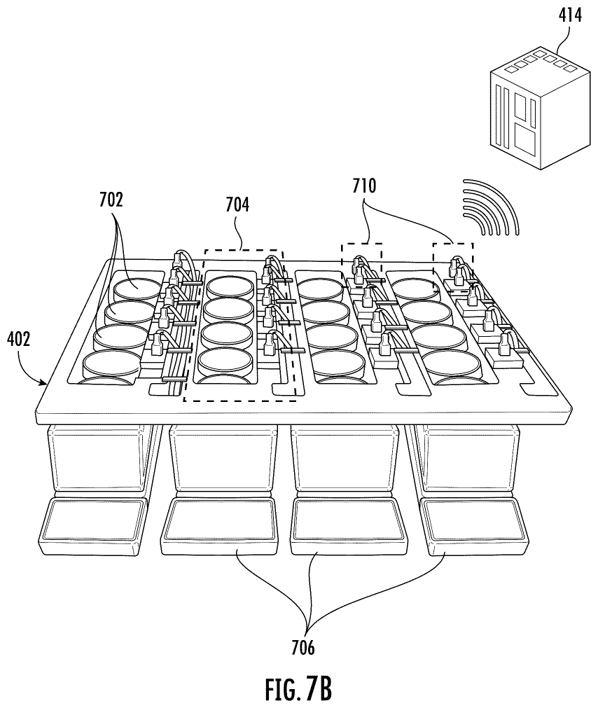

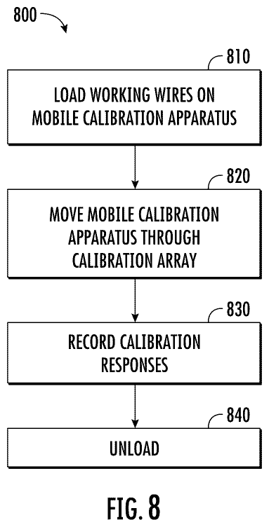

FIG. 4A is a perspective view and FIG. 4B is a top view of a calibration system 400 for calibrating the working wire 100 of a biological sensor (and thus for calibrating the biological sensor), in accordance with some examples. The calibration system 400 may be used within an overall manufacturing facility for manufacturing the biological sensors and generally includes calibration arrays 402, fixture trays 404, and a robot assembly 406 within a housing. Several calibration arrays 402 are shown in this example. Each calibration array 402 comprises a plurality of containers (more clearly shown below with respect to FIGS. 7A and 7B) configured to hold calibration solutions during the calibration process. In various examples, any number of the calibration arrays 402 may be included, such as one, two, four or more calibration arrays. The fixture trays 404 may be placed or slid into holding areas of the calibration system 400. The fixture trays 404 hold multiple fixture/apparatus combinations to be tested or calibrated. Two of the fixture trays 404 are shown in this example, but any appropriate number may be used. The robot assembly 406 is configured to transport the fixture/apparatus combinations from the fixture trays 404 to the plurality of containers in the calibration arrays 402 for testing or calibrating.

Additionally, a top peripheral barrier 408 is shown in FIG. 4A around the top of the housing of the calibration system 400 and surrounding and partially obscuring the area where the robot assembly 406 moves. The top peripheral barrier 408 protects the robot assembly 406 from being inadvertently touched or contacted by system users and protects the system users from a potential impact by the robot assembly 406. Furthermore, side panels 410 with transparent doors and/or windows may be provided around the portions of the housing of the calibration system 400 where the calibration arrays 402 and the fixture trays 404 are positioned. The side panels 410 thus prevent unintentional access to or contamination of the calibration arrays 402 and the fixture trays 404, while allowing appropriate access by the system users for inserting and removing the calibration arrays 402 and the fixture trays 404 into and out of the housing of the calibration system 400 as needed during the calibration process. In addition, a control panel 412 on the exterior of the housing and control electronics 414 within the housing (such as a computerized system) are provided for controlling and monitoring the calibration process. For example, the system user can operate the control panel 412 to turn the calibration system 400 on and off and to set operating parameters for the calibration process.

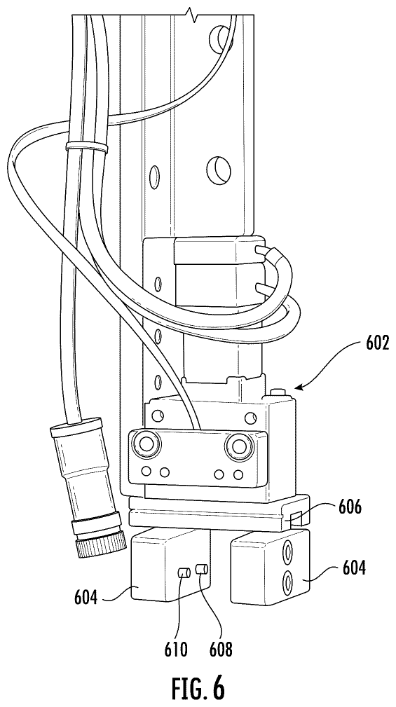

The robot assembly 406 (e.g., a robotic mechanism with a robotic manipulation arm) is shown as a gantry robot in this example, shown in FIG. 4C. The gantry robot enables a wide range of motion in X, Y and Z directions on an X-direction track 416, a Y-direction track 418, and a Z-direction track 420 in the calibration system 400. The tracks are mounted on the top of the calibration system 400, so that clamping jaws (described below with reference to FIG. 6) can be raised and lowered from above to pick up, transport, and place the fixture/apparatus combinations to and from the calibration arrays 402 and the fixture trays 404. In other examples, the robot assembly may be one or more stationary 6-axis robots located at one or more appropriate positions within the calibration system 400 to move the wire-holding fixtures within a designated range. For example, one stationary robot may be included for each calibration array 402, to move fixtures between that array and a fixture tray where mobile calibration apparatuses 300 are brought to or removed from the calibration system.

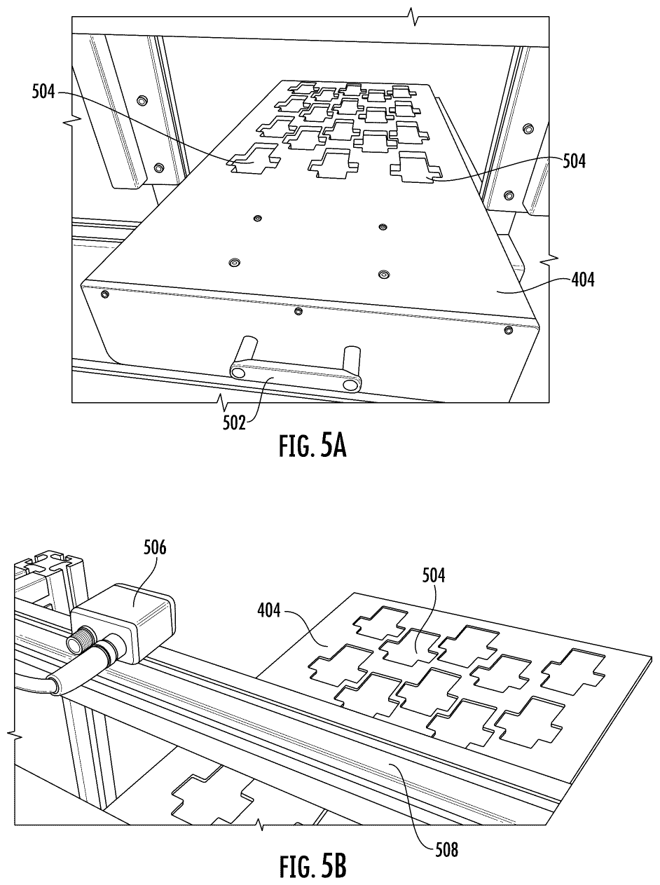

FIG. 5A is a perspective view of one of the fixture trays 404 in the holding area. The fixture tray 404 has a handle 502 with which the system user can push and pull the fixture tray 404 into and out of the holding area. In this manner, the mobile calibration apparatuses 300 can be transferred into and out of the calibration system 400. The working wires 100 on the wire-holding fixtures 200 coupled to the mobile calibration apparatuses 300 are stored or staged in the fixture tray 404 while awaiting processing within the calibration system 400 or transport to another manufacturing station in the overall manufacturing facility.

The fixture tray 404 has multiple apertures 504 shaped according to the outline of the mobile calibration apparatus 300 (or the fixture/apparatus combination), in this example. In other examples, the holding area may have racks or jigs configured to hold the mobile calibration apparatuses 300. In some examples, each fixture/apparatus combination is placed in a respective aperture 504 with the handle 310 of the mobile calibration apparatus 300 pointed upward, so that the robotic manipulation arm can pick up the fixture/apparatus combination. Consequently, the wire-holding fixtures 200 will be disposed on the underside of the mobile calibration apparatus 300 with the working wires 100 extending downwards.

The fixture tray 404 is a sliding drawer in this example, to bring the wire-holding fixtures 200 into or remove the wire-holding fixtures 200 out of the calibration system 400, either manually by the system user or in an automated manner (e.g., with robots moving them from a previous manufacturing station and/or to a subsequent manufacturing station within the overall manufacturing facility). In other examples, other holding area configurations instead of the fixture trays 404 are possible, such as a conveyor to bring the mobile calibration apparatuses 300 to or from the calibration system 400.

FIG. 5B shows an optical device 506 (e.g., a camera, laser scanner, or RFID reader (if RFID tags are used)) installed (e.g., mounted on a rail 508) near the fixture tray 404 in or adjacent to the holding area. The optical device 506 is positioned to scan the identifier 204 on the wire-holding fixtures 200 to track the location of the wire-holding fixtures 200 by reading their QR codes (or other scannable code). The optical device 506 enables manufacturing data to be recorded for the working wires 100 (e.g., on a computer processor or controller in communication with the calibration system 400), such as the wire-holding fixture 200 having completed the calibration process, and the calibration results for each working wire 100 in the wire-holding fixture 200. In this illustration, the optical device 506 is positioned near and in view of the fixture tray 404 so that as the robot assembly 406 picks up a fixture/apparatus combination from a fixture tray 404, it can move the fixture/apparatus combination in front of the optical device 506 for the identifiers 204 on the wire-holding fixtures 200 to be scanned. Additional optical devices may be included at other locations, such as near each calibration array 402 and/or on the robot assembly 406 so that the calibration system 400 (e.g., via the control electronics 414, which may be a computer processor or controller) can instruct the robot assembly 406 where to move that particular wire-holding fixture 200 to proceed through the calibration process within the calibration system 400. The control electronics 414 may also determine if any of the working wires 100 is out of specification (e.g., does not meet operating parameters), in which case the wire-holding fixture 200 can be removed from the process rather than continuing through the remainder of the calibration cycle.

FIG. 6 is a perspective view of a robotic manipulation arm 602 that may be part of the robot assembly 406 of FIG. 4C, in accordance with some examples. In this example, two clamps (e.g., clamping jaws 604) slide on a rail 606 (linear motor) so that they can move toward each other to grip the mobile calibration apparatus 300 and away from each other to release the mobile calibration apparatus 300. The clamping jaws 604 are flat in this example and lined with a gripping material, such as neoprene (i.e., polychloroprene, or other medical grade materials) to securely grab the handle 310 of the mobile calibration apparatus 300 of FIG. 3A. Additionally, alignment pins 608 and 610 are schematically shown, with diameters different from each other corresponding to the alignment features 312 and 314, respectively, shown in FIG. 3A. The alignment pins 608 and 610 and alignment features 312 and 314 orient the mobile calibration apparatus 300 in the proper direction and keep the wire-holding fixtures 200 stable in a reference plane to the robot assembly 406 so that the electrical contacts 308 are properly seated. For example, the alignment pins 608 and 610 and alignment features 312 and 314 help ensure that the pogo pins of the electrical contacts 308 will not be broken while the mobile calibration apparatus 300 is being moved through the calibration system 400, and/or that the pogo pins stay connected to the metal strips 216 for the correct working wires in the wire-holding fixture 200.

FIGS. 7A and 7B are perspective views of one of the calibration arrays 402 shown above in FIGS. 4A and 4B, in accordance with some examples. The calibration array includes a plurality of containers 702, such as dishes or beakers, configured to hold calibration solutions. The calibration solutions may be, for example, various enzymes, an electrochemical break-in solution (e.g., phosphate buffer solution “PBS”), an interferent solution (e.g., containing acetaminophen), or various concentrations of glucose. The containers 702 are arranged in rows 704 (or banks) and columns, with multiple containers 702 in each row in this example. In other examples, the containers 702 may be arranged in other configurations, such as a single row. When the robot assembly 406 moves one of the fixture/apparatus combinations to one of the containers 702 (i.e., a calibration station), the fixture/apparatus combination is placed onto or above the container 702 with the working wires 100 extending downward into the calibration solution, so that the testing or calibrating can be performed for this calibration solution. The robot assembly 406 thus transports the fixture/apparatus combination through a sequence of the containers 702, such that the fixture/apparatus combination is sequentially placed on or above each container 702 in the sequence with the working wires 100 extending into the calibration solution for the calibration process.

A hotplate 706 is underneath each container 702 (or one hot plate under a whole row of containers) to keep the calibration solutions at a desired temperature (e.g., a typical human body temperature of about 37° C.+/−2° C.), such as for the glucose and interferent solutions. In this example, the hotplates 706 each have multiple heating positions, such that multiple containers 702 (e.g., of a row) are positioned along each hotplate 706. Using one hotplate 706 for multiple containers 702 advantageously helps ensure that all the solutions in that row 704 of containers 702 have the same temperature. Different temperatures between calibration solutions, on the other hand, can cause a temperature offset which will affect the linearity and the slope of the electrical current response for the biological sensor, and consequently the accuracy of the calculated sensitivity (typically measured in nanoamperes per millimolar or micromolar of glucose level; nA/mM or nA/μM). In some examples, a thermally conductive plate, such as an aluminum plate, may be placed on the hotplate 706 for the containers 702 to be placed on. The thermally conductive plate can help ensure a uniform temperature across the heating surfaces of the hotplate 706 and all the containers 702 on that hotplate 706.

The robot assembly 406 of the calibration system 400 moves the wire-holding fixtures 200 (i.e., the fixture/apparatus combinations) through a sequence of the containers 702 and calibration solutions for a series of tests in the calibration array 402. For example, the sequence may involve containers 702 of a break-in solution (e.g., PBS), an interferent solution, various levels of glucose, and then a low oxygen solution (e.g., 0.4 mmHg partial pressure of oxygen) in this order or any other appropriate order. In some examples, multiple containers 702 of PBS may be in the calibration array 402 to account for the longer time needed for the break-in period compared to the times needed for the other solutions in the other containers 702. For example, if the working wires 100 are submerged for 5 minutes in each container 702 in a sequence, then six containers 702 of PBS will provide 30 minutes of electrochemical break-in. Following this, one container 702 of each (e.g., 5 minutes of each) of the interferent, glucose concentrations, and low oxygen solutions may be utilized to perform the calibrations for each of those conditions. For example, the working wires 100 of each wire-holding fixture 200 may be sequentially dipped in multiple containers 702 of PBS, then one container 702 of an interferent solution (e.g., a supra-physiologic dose of acetaminophen), one container 702 of 40 mg/dl glucose, one container 702 of 100 mg/dl glucose, one container 702 of 200 mg/dl glucose, one container 702 of 400 mg/dl glucose, and one container 702 of low oxygen. Alternatively, for calibrating lactate monitors (i.e., lactate sensors) the solutions in containers 702 may be lactate solutions of appropriate concentrations, e.g., 1, 2, 5 or 10 mM (i.e., approximately 0.06 mg/dl, 0.11 mg/dl, 0.28 mg/dl or 0.56 mg/dl). FIG. 7A also shows a container of water (water container 708) which may be used at the completion of the entire calibration cycle to wash off glucose and/or other solutions from the working wires 100, so that the glucose or other solutions do not degrade the membrane materials of the working wires 100 of the biological sensor.

In FIG. 7B, electrical components 710 for each calibration station (i.e., container 702) can also be seen. These electrical components 710 include wiring for secondary electronics, such as wireless communications to transmit data generated from the working wires 100 being calibrated at that station, and temporary data storage. The data can be transmitted to the control electronics 414 (or other controller or computer processor) for the calibration system 400 to store and process the calibration data. The calibration data and correlated sensitivities, using manufacturing data for each working wire 100 (e.g., coating layer thicknesses and manufacturing conditions), can be stored and communicated to a finished biological sensor device to supply accurate calibration for each particular biological sensor.

The calibration system 400 is scalable by including more calibration stations, such as more calibration arrays 402, containers 702, fixture trays 404, and/or robot assemblies 406. Data from the calibration tests of all the working wires 100 being processed can be analyzed to create factory calibration curves that accurately predict in vivo values for the working wires 100. For example, because each individual wire-holding fixture 200 (and consequently each working wire 100, by recording data therefor) is tracked by the identifier 204, the calibration responses can be correlated with the manufacturing parameters for each individual working wire 100 (e.g., layer thicknesses for each membrane in the working wire 100, and the manufacturing conditions for each layer). Manufacturing conditions that produce certain sensor responses can be analyzed and aggregated and used to provide a sensitivity for that particular working wire 100 when used in the field, thus reducing the need for manual finger stick calibration by users.

FIG. 8 is a flowchart of a calibration process 800 for calibrating the working wires 100 for a biological sensor (and thus for calibrating the biological sensor) in an automated manner, in accordance with some examples. In block 810, the working wires are loaded onto the mobile calibration apparatus 300. The working wires 100 may be on a wire-holding fixture 200 (e.g., FIGS. 2A-2B) that is coupled to the mobile calibration apparatus 300. In block 820, the mobile calibration apparatus 300 is moved through one or more of the calibration arrays 402. The movement may be performed by a robotic mechanism, such as the robot assembly 406. The calibration array 402 may include a plurality of containers 702 containing various calibration solutions such as an electrochemical break-in solution, an interferent solution, various levels of glucose concentration solutions (or lactate concentration solutions), and a low oxygen solution. In block 830, as the working wires 100 are subjected to each calibration solution, calibration responses are recorded (by the control electronics 414) for the working wires 100 with respect to each calibration solution. In block 840, after the working wires 100 have moved through the entire calibration array 402, they are unloaded from the calibration system 400. The unloading may involve removing the mobile calibration apparatus 300 from the calibration system 400 and/or removing the working wires 100 from the mobile calibration apparatus 300. After this calibration process, the biological sensors can be calibrated based on the recorded calibration responses of the respective working wires that are incorporated into the biological sensors.

As described herein, the present systems and processes enable calibration of a working wire of a biological sensor, such as a glucose sensor, to be performed in an automated fashion with high accuracy and trackability.

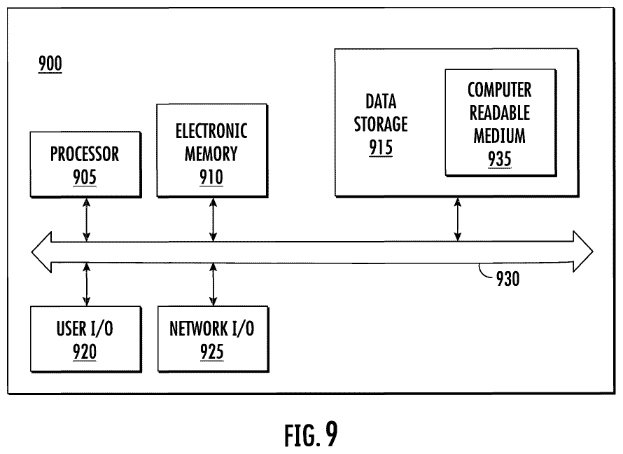

FIG. 9 is a simplified schematic diagram showing an example computer (or server) 900 (representing any combination of one or more of computers) for use in the control electronics 414 of the calibration system 400, in accordance with some examples. Other examples may use other components and combinations of components. For example, the computer 900 may represent one or more physical computer devices or servers, such as web servers, rack-mounted computers, network storage devices, desktop computers, laptop/notebook computers, etc., depending on the complexity. In some examples implemented at least partially in a cloud network potentially with data synchronized across multiple geolocations, the computer 900 may be referred to as one or more cloud servers. In some examples, the functions of the computer 900 are enabled in a single computer device. In more complex implementations, some of the functions of the computing system are distributed across multiple computer devices, whether within a single server farm facility or multiple physical locations. In some examples, the computer 900 functions as a single virtual machine.

In the illustrated example, the computer 900 generally includes at least one processor 905, a main electronic memory 910, a data storage 915, a user input/output (I/O) 920, and a network I/O 925, among other components not shown for simplicity, connected or coupled together by a data communication subsystem 930. A non-transitory computer readable medium 935 includes instructions that, when executed by the processor 905, cause the processor 905 to perform operations including calculations and methods as described herein.

In accordance with the description herein, the various components of the system or method generally represent appropriate hardware and software components for providing the described resources and performing the described functions. The hardware generally includes any appropriate number and combination of computing devices, network communication devices, and peripheral components connected together, including various processors, computer memory (including transitory and non-transitory media), input/output devices, user interface devices, communication adapters, communication channels, etc. The software generally includes any appropriate number and combination of conventional and specially-developed software with computer-readable instructions stored by the computer memory in non-transitory computer-readable or machine-readable media and executed by the various processors to perform the functions described herein.

Any method (also referred to as a “process” or an “approach”) described or otherwise enabled by the disclosure herein may be implemented by hardware components (e.g., machines), software modules (e.g., stored in machine-readable media), or a combination thereof. By way of example, machines may include one or more computing device(s), processor(s), controller(s), integrated circuit(s), chip(s), system(s) on a chip, server(s), programmable logic device(s), field programmable gate array(s), electronic device(s), special purpose circuitry, and/or other suitable device(s) described herein or otherwise known in the art. One or more non-transitory machine-readable media embodying program instructions that, when executed by one or more machines, cause the one or more machines to perform or implement operations comprising the steps of any of the methods described herein are contemplated herein. As used herein, machine-readable media includes all forms of machine-readable media (e.g., one or more non-volatile or volatile storage media, removable or non-removable media, integrated circuit media, magnetic storage media, optical storage media, or any other storage media, including RAM, ROM, and EEPROM) that may be patented under the laws of the jurisdiction in which this application is filed, but does not include machine-readable media that cannot be patented under the laws of the jurisdiction in which this application is filed.

Systems that include one or more machines and one or more non-transitory machine-readable media are also contemplated herein. One or more machines that perform or implement, or are configured, operable, or adapted to perform or implement operations comprising the steps of any methods described herein are also contemplated herein. Method steps described herein may be order independent and can be performed in parallel or in an order different from that described if possible to do so. Different method steps described herein can be combined to form any number of methods, as would be understood by one of ordinary skill in the art. Any method step or feature disclosed herein may be omitted from a claim for any reason. Certain well-known structures and devices are not shown in figures to avoid obscuring the concepts of the present disclosure. When two things are “coupled to” each other, those two things may be directly connected together, or separated by one or more intervening things. Where no lines or intervening things connect two particular things, coupling of those things is contemplated in at least one example unless otherwise stated. Where an output of one thing and an input of another thing are coupled to each other, information sent from the output is received in its outputted form or a modified version thereof by the input even if the information passes through one or more intermediate things. Any known communication pathways and protocols may be used to transmit information (e.g., data, commands, signals, bits, symbols, chips, and the like) disclosed herein unless otherwise stated. The words comprise, comprising, include, including and the like are to be construed in an inclusive sense (i.e., not limited to) as opposed to an exclusive sense (i.e., consisting only of). Words using the singular or plural number also include the plural or singular number, respectively, unless otherwise stated. The word “or” and the word “and” as used in the Detailed Description cover any of the items and all of the items in a list unless otherwise stated. The words some, any and at least one refer to one or more. The terms may or can are used herein to indicate an example, not a requirement—e.g., a thing that may or can perform an operation, or may or can have a characteristic, need not perform that operation, or have that characteristic in each example, but that thing performs that operation or has that characteristic in at least one example. Unless an alternative approach is described, access to data from a source of data may be achieved using known techniques (e.g., requesting component requests the data from the source via a query or other known approach, the source searches for and locates the data, and the source collects and transmits the data to the requesting component, or other known techniques).

Example aspects of the present systems and methods are described in the clauses below.

### CLAUSES

Clause 1. An apparatus for calibrating a biological sensor, comprising: a housing; a holder coupled to the housing, the holder configured to retain a wire-holding fixture for holding a working wire for the biological sensor; and an electrical test board coupled to the housing, wherein the electrical test board is configured with electronics of a continuous biological monitor and has an electrical contact configured to electrically connect to the working wire through the wire-holding fixture.

Clause 2. The apparatus of clause 1, further comprising a battery in the housing, the battery electrically connected to the electrical test board for providing electrical power to the electrical test board and the working wire throughout a calibration process.

Clause 3. The apparatus of clause 1, further comprising an alignment feature configured to be gripped by a robotic manipulation arm.

Clause 4. The apparatus of clause 3, further comprising a handle coupled to the housing, wherein the handle has the alignment feature.

Clause 5. The apparatus of clause 1, wherein: the holder is configured to retain a plurality of wire-holding fixtures; and each wire-holding fixture is configured to hold a plurality of working wires for a corresponding plurality of biological sensors.

Clause 6. The apparatus of clause 1, wherein: the working wire extends away from the wire-holding fixture and is configured to be able to extend into a calibration solution in a container during a calibration process.

Clause 7. A system for calibrating a biological sensor, comprising: a plurality of containers configured to hold calibration solutions; a mobile calibration apparatus configured to be placed on the containers; a wire-holding fixture for holding a working wire for the biological sensor; and a robot assembly configured to transport a combination of the mobile calibration apparatus and the wire-holding fixture to and from the plurality of containers for a calibration process; wherein the mobile calibration apparatus comprises: a housing; a holder coupled to the housing, the holder configured to hold the wire-holding fixture; and an electrical test board coupled to the housing, wherein the electrical test board is configured with electronics of a continuous biological monitor and has an electrical contact configured to electrically connect to the working wire through the wire-holding fixture.

Clause 8. The system of clause 7, wherein the robot assembly is a gantry robot.

Clause 9. The system of clause 7, further comprising an optical device positioned to scan a scannable code on the wire-holding fixture.

Clause 10. The system of clause 7, wherein the mobile calibration apparatus further comprises a battery in the housing, the battery electrically connected to the electrical test board for providing electrical power to the electrical test board and the working wire throughout the calibration process.

Clause 11. The system of clause 7, wherein the mobile calibration apparatus further comprises an alignment feature configured to be gripped by a robotic manipulation arm of the robot assembly.

Clause 12. The system of clause 11, wherein the mobile calibration apparatus further comprises a handle coupled to the housing, wherein the handle has the alignment feature.

Clause 13. The system of clause 7, wherein: the holder is configured to retain a plurality of wire-holding fixtures; and each wire-holding fixture is configured to hold a plurality of working wires for a corresponding plurality of biological sensors.

Clause 14. The system of clause 7, wherein: the working wire extends away from the wire-holding fixture and is configured to be able to extend into the calibration solutions in the containers during the calibration process.

Clause 15. A method comprising: providing a combination of a mobile calibration apparatus and a wire-holding fixture, wherein the wire-holding fixture holds a working wire for a biological sensor; transporting, by a robot assembly, the combination of the mobile calibration apparatus and the wire-holding fixture through a sequence of containers containing calibration solutions, wherein the combination of the mobile calibration apparatus and the wire-holding fixture is sequentially placed above each container in the sequence with the working wire extending into the calibration solution; testing the working wire with the calibration solutions; recording calibration responses of the working wire with respect to the calibration solutions; and calibrating the biological sensor based on the recorded calibrated responses; wherein the mobile calibration apparatus comprises: a housing; a holder coupled to the housing, the holder configured to hold the wire-holding fixture; and an electrical test board coupled to the housing, wherein the electrical test board is configured with electronics of a continuous biological monitor and has an electrical contact configured to electrically connect to the working wire through the wire-holding fixture.

Clause 16. The method of clause 15, further comprising: providing, by a battery disposed in the housing and electrically connected to the electrical test board and the working wire, continuous electrical power to the electrical test board and the working wire during a time in which the combination of the mobile calibration apparatus and the wire-holding fixture is transported through the sequence of containers.

Clause 17. The method of clause 15, wherein the transporting of the combination of the mobile calibration apparatus and the wire-holding fixture comprises: gripping an alignment feature of the mobile calibration apparatus by a robotic manipulation arm of the robot assembly.

Clause 18. The method of clause 17, wherein the mobile calibration apparatus further comprises a handle coupled to the housing, wherein the handle has the alignment feature.

Clause 19. The method of clause 15, wherein: the holder is configured to retain a plurality of wire-holding fixtures; and each wire-holding fixture is configured to hold a plurality of working wires for a corresponding plurality of biological sensors.

Clause 20. The method of clause 15, wherein: the working wire extends away from the wire-holding fixture and is configured to be able to extend into the calibration solutions in the containers when placed above the containers.

Reference has been made in detail to examples of the disclosed invention, one or more examples of which have been illustrated in the accompanying figures. Each example has been provided by way of explanation of the present technology, not as a limitation of the present technology. In fact, while the specification has been described in detail with respect to specific examples of the invention, it will be appreciated that those skilled in the art, upon attaining an understanding of the foregoing, may readily conceive of alterations to, variations of, and equivalents to these examples. For instance, features illustrated or described as part of one example may be used with another example to yield a still further example. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. An apparatus for calibrating a biological sensor, comprising:
a housing;
a holder coupled to the housing, the holder configured to retain a wire-holding fixture for holding a working wire for the biological sensor; and
an electrical test board coupled to the housing, wherein the electrical test board is configured with electronics of a continuous biological monitor and has an electrical contact configured to electrically connect to the working wire through the wire-holding fixture.

2. The apparatus of claim 1, further comprising a battery in the housing, the battery electrically connected to the electrical test board for providing electrical power to the electrical test board and the working wire throughout a calibration process.

3. The apparatus of claim 1, further comprising an alignment feature configured to be gripped by a robotic manipulation arm.

4. The apparatus of claim 3, further comprising a handle coupled to the housing, wherein the handle has the alignment feature.

5. The apparatus of claim 1, wherein:
the holder is configured to retain a plurality of wire-holding fixtures; and
each wire-holding fixture is configured to hold a plurality of working wires for a corresponding plurality of biological sensors.

6. The apparatus of claim 1, wherein:
the working wire extends away from the wire-holding fixture and is configured to be able to extend into a calibration solution in a container during a calibration process.

7. A system for calibrating a biological sensor, comprising:
a plurality of containers configured to hold calibration solutions;
a mobile calibration apparatus configured to be placed on the containers;
a wire-holding fixture for holding a working wire for the biological sensor; and
a robot assembly configured to transport a combination of the mobile calibration apparatus and the wire-holding fixture to and from the plurality of containers for a calibration process;
wherein the mobile calibration apparatus comprises:
a housing;
a holder coupled to the housing, the holder configured to hold the wire-holding fixture; and
an electrical test board coupled to the housing, wherein the electrical test board is configured with electronics of a continuous biological monitor and has an electrical contact configured to electrically connect to the working wire through the wire-holding fixture.

8. The system of claim 7, wherein the robot assembly is a gantry robot.

9. The system of claim 7, further comprising an optical device positioned to scan a scannable code on the wire-holding fixture.

10. The system of claim 7, wherein the mobile calibration apparatus further comprises a battery in the housing, the battery electrically connected to the electrical test board for providing electrical power to the electrical test board and the working wire throughout the calibration process.

11. The system of claim 7, wherein the mobile calibration apparatus further comprises an alignment feature configured to be gripped by a robotic manipulation arm of the robot assembly.

12. The system of claim 11, wherein the mobile calibration apparatus further comprises a handle coupled to the housing, wherein the handle has the alignment feature.

13. The system of claim 7, wherein:
the holder is configured to retain a plurality of wire-holding fixtures; and
each wire-holding fixture is configured to hold a plurality of working wires for a corresponding plurality of biological sensors.

14. The system of claim 7, wherein:
the working wire extends away from the wire-holding fixture and is configured to be able to extend into the calibration solutions in the containers during the calibration process.

15. A method comprising:
providing a combination of a mobile calibration apparatus and a wire-holding fixture, wherein the wire-holding fixture holds a working wire for a biological sensor;
transporting, by a robot assembly, the combination of the mobile calibration apparatus and the wire-holding fixture through a sequence of containers containing calibration solutions, wherein the combination of the mobile calibration apparatus and the wire-holding fixture is sequentially placed above each container in the sequence with the working wire extending into the calibration solution;
testing the working wire with the calibration solutions;
recording calibration responses of the working wire with respect to the calibration solutions; and
calibrating the biological sensor based on the recorded calibrated responses;
wherein the mobile calibration apparatus comprises: a housing; a holder coupled to the housing, the holder configured to hold the wire-holding fixture; and an electrical test board coupled to the housing, wherein the electrical test board is configured with electronics of a continuous biological monitor and has an electrical contact configured to electrically connect to the working wire through the wire-holding fixture.

16. The method of claim 15, further comprising:
providing, by a battery disposed in the housing and electrically connected to the electrical test board and the working wire, continuous electrical power to the electrical test board and the working wire during a time in which the combination of the mobile calibration apparatus and the wire-holding fixture is transported through the sequence of containers.

17. The method of claim 15, wherein the transporting of the combination of the mobile calibration apparatus and the wire-holding fixture comprises:
gripping an alignment feature of the mobile calibration apparatus by a robotic manipulation arm of the robot assembly.

18. The method of claim 17, wherein the mobile calibration apparatus further comprises a handle coupled to the housing, wherein the handle has the alignment feature.

19. The method of claim 15, wherein:
the holder is configured to retain a plurality of wire-holding fixtures; and
each wire-holding fixture is configured to hold a plurality of working wires for a corresponding plurality of biological sensors.

20. The method of claim 15, wherein:
the working wire extends away from the wire-holding fixture and is configured to be able to extend into the calibration solutions in the containers when placed above the containers.
